(19) United States
(12) Patent Application Publication
(10) Pub. No.: US 2026/0027520 A1
(43) Pub. Date: Jan. 29, 2026

(54) ETHYLENE OXIDE ABATEMENT SYSTEMS

(71) Applicant: Boston Scientific Scimed, Inc., Maple Grove, MN (US)

(72) Inventors: Joseph Thomas Delaney, Jr., Minneapolis, MN (US); Stephen Yeadon, Salem, SC (US); Nicolas Ball-Jones, Minneapolis, MN (US); Lauren Sfakis Lydecker, Upton, MA (US); Vishal Bavishi, Natick, MA (US)

(73) Assignee: Boston Scientific Scimed, Inc., Maple Grove, MN (US)

(21) Appl. No.: 19/280,225

(22) Filed: Jul. 25, 2025

Related U.S. Application Data
(60) Provisional application No. 63/675,390, filed on Jul. 25, 2024.

(51) Int. Cl. B01D 53/82; A61L 2/20; A61L 2/26; B01D 53/72; B01J 20/24; B01J 20/28; B65D 81/18

(52) U.S. Cl. CPC B01D 53/82; A61L 2/206; A61L 2/26; B01D 53/72; B01J 20/24; B01J 20/2805; B65D 81/18; A61L 2202/13; A61L 2202/24; B01D 2251/70; B01D 2251/80; B01D 2253/202; B01D 2257/708

(57) ABSTRACT

Systems for abatement of sterilizing gas are disclosed. An example system may include a medical device package having a medical device disposed therein. A sterilizing gas abatement member may be disposed adjacent to the medical device. The sterilizing gas abatement member may be configured to abate a sterilizing gas present adjacent to the medical device.

### CROSS-REFERENCE TO RELATED APPLICATIONS

This application claims the benefit of priority under 35 U.S.C. § 119 of U.S. Provisional Application No. 63/675,390, filed Jul. 25, 2024, the entire disclosure of which is hereby incorporated by reference.

### TECHNICAL FIELD

The present disclosure pertains to medical devices, and methods for manufacturing and packaging medical devices. More particularly, the present disclosure pertains to ethylene oxide abatement systems.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing, packaging, and using medical devices.

### BRIEF SUMMARY

This disclosure provides design, material, manufacturing and packaging methods, and use alternatives for medical devices. A system for abatement of sterilizing gas is disclosed. The system comprises: a medical device package having a medical device disposed therein; and a sterilizing gas abatement member disposed adjacent to the medical device, the sterilizing gas abatement member being configured to abate a sterilizing gas present adjacent to the medical device.

Alternatively or additionally to any of the embodiments above, the medical device comprises a sterilized medical device.

Alternatively or additionally to any of the embodiments above, the sterilizing gas comprises ethylene oxide.

Alternatively or additionally to any of the embodiments above, the sterilizing gas abatement member includes a sachet.

Alternatively or additionally to any of the embodiments above, the sachet is disposed within the medical device package.

Alternatively or additionally to any of the embodiments above, the medical device package includes an inner pouch and an outer pouch, and wherein the sachet is disposed within the inner pouch.

Alternatively or additionally to any of the embodiments above, the medical device package includes an inner pouch and an outer pouch, and wherein the sachet is disposed within the outer pouch.

Alternatively or additionally to any of the embodiments above, the sterilizing gas abatement member includes a blanket.

Alternatively or additionally to any of the embodiments above, the blanket is disposed about the medical device package.

Alternatively or additionally to any of the embodiments above, further comprising one or more additional medical device packages, and wherein the blanket is disposed about the medical device package and the one or more additional medical device packages.

Alternatively or additionally to any of the embodiments above, further comprising a sterilizing gas abatement substance incorporated within the sterilizing gas abatement member.

Alternatively or additionally to any of the embodiments above, the sterilizing gas abatement substance includes a hydrophilic material.

Alternatively or additionally to any of the embodiments above, the sterilizing gas abatement substance includes a hydrophilic polymer.

Alternatively or additionally to any of the embodiments above, the sterilizing gas abatement substance includes a nucleophilic material.

Alternatively or additionally to any of the embodiments above, the sterilizing gas abatement substance includes chitosan.

Alternatively or additionally to any of the embodiments above, the sterilizing gas abatement substance includes a lignosulfonic acid.

A system for abatement of residual ethylene oxide is disclosed. The system comprises: a medical device package having a sterilized medical device disposed therein; wherein the medical device package includes an inner package, an outer package, or both; an ethylene oxide abatement sachet disposed adjacent to the sterilized medical device, the sachet having an ethylene oxide abatement substance disposed therein; and wherein the sachet is configured to abate residual ethylene oxide present adjacent to the sterilized medical device.

Alternatively or additionally to any of the embodiments above, the ethylene oxide abatement substance includes a hydrophilic material, a hydrophilic polymer, a nucleophilic material, chitosan, a lignosulfonic acid, or combinations thereof.

A system for abatement of residual ethylene oxide is disclosed. The system comprises: a medical device package having a sterilized medical device disposed therein; wherein the medical device package includes an inner package, an outer package, or both; an ethylene oxide abatement blanket disposed adjacent to the sterilized medical device, the blanket having an ethylene oxide abatement substance incorporated therein; and wherein the blanket is configured to abate residual ethylene oxide present adjacent to the sterilized medical device.

Alternatively or additionally to any of the embodiments above, the ethylene oxide abatement substance includes a hydrophilic material, a hydrophilic polymer, a nucleophilic material, chitosan, a lignosulfonic acid, or combinations thereof.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically depicts an example medical device disposed within a medical device package.

FIG. 2 schematically depicts an example medical device disposed within a medical device package and with a sterilizing gas abatement member disposed within the medical device package.

FIG. 3 schematically depicts an example medical device disposed within a medical device package and with a sterilizing gas abatement member disposed within the medical device package.

FIG. 4 schematically depicts an example medical device disposed within a medical device package and with a sterilizing gas abatement member disposed within the medical device package.

FIG. 5 schematically depicts a plurality of medical device packages with a sterilizing gas abatement member disposed within the medical device packages.

FIG. 6 schematically depicts a plurality of medical device packages.

FIG. 7 schematically depicts a plurality of medical device packages with a sterilizing gas abatement member disposed within the medical device packages.

FIG. 8 schematically depicts a plurality of medical device packages.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

Medical devices such as guidewires, catheters, balloon catheters, stents, stent delivery systems, and the like are sterilized prior to use. A number of different sterilization techniques may be used. One example sterilization technique is the use of ethylene oxide gas. While effective for sterilization, residual ethylene oxide gas can be present (e.g., off-gas) from the devices and/or packaging. In general, the relative amount of residual ethylene oxide gas present may be considered low. However, in a manufacturing setting where relatively large quantities of sterilized and packaged medical devices may be present, the levels of residual ethylene oxide gas can rise to undesirable levels. Disclosed herein are systems for abatement of sterilizing gas, for example ethylene oxide.

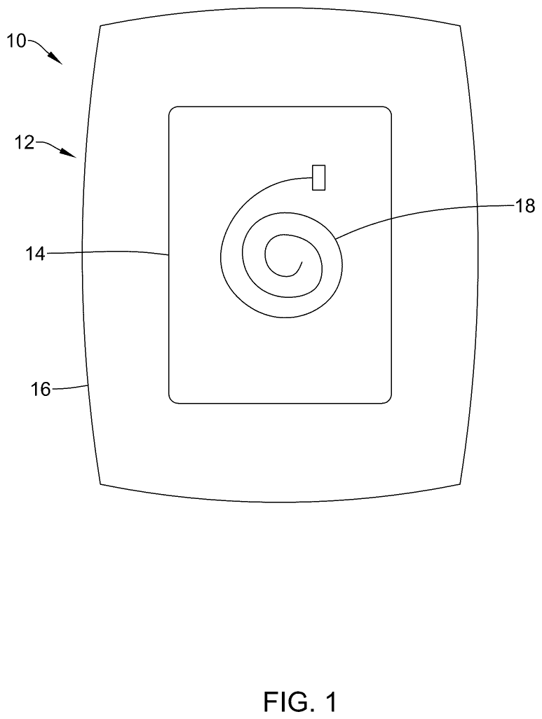

FIG. 1 schematically depicts an example system 10. The system 10 may include a medical device package 12. In this example, the medical device package 12 may include an inner packaging member or pouch 14 and an outer packaging member or pouch 16. Examples are contemplated that utilize more or fewer components. A medical device 18, depicted schematically, may be disposed within the medical device package 12. The medical device 18 may be any of a variety of different medical devices such as guidewires, catheters, balloon catheters, stents, stent delivery systems, and the like. These are just examples. Other forms of medical devices are contemplated. In this example, the medical device 18 is shown disposed within the inner packaging member/pouch 14.

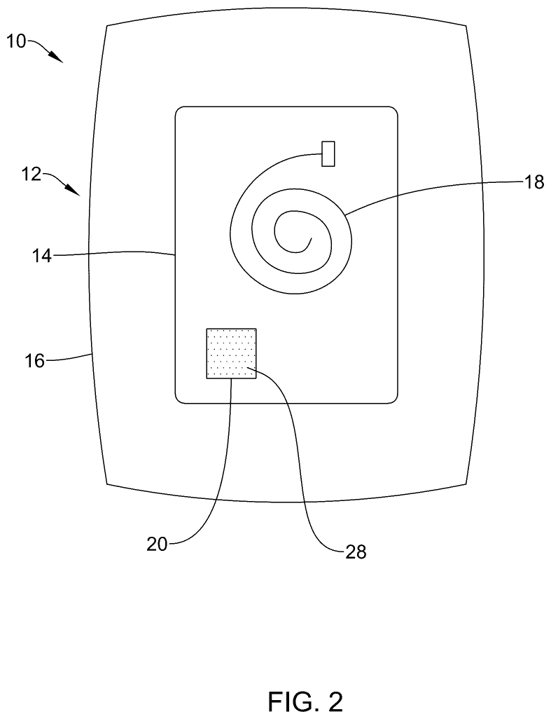

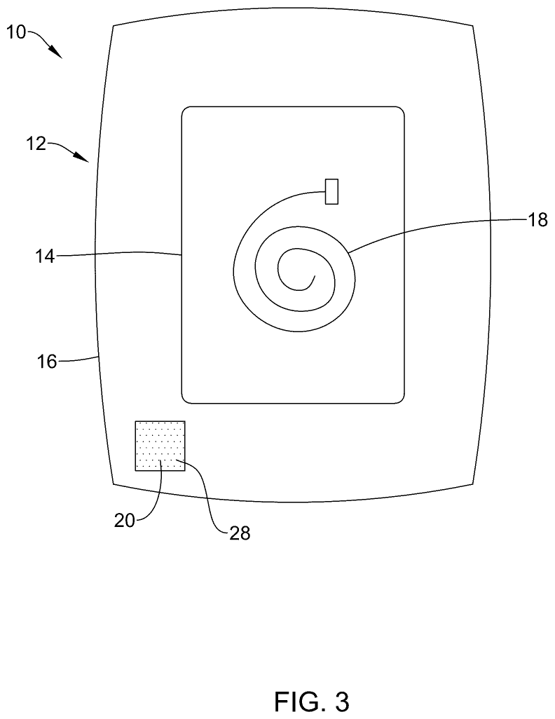

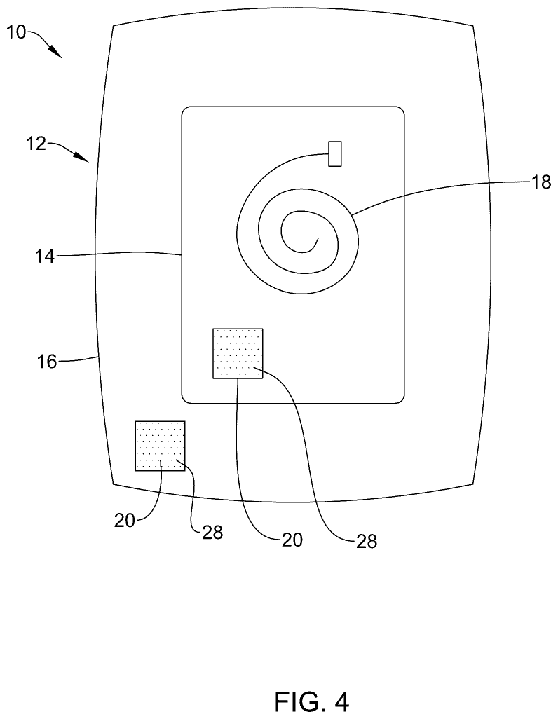

As indicated herein, medical devices like the medical device 18 may be sterilized to eliminate the presence of microorganisms and to provide efficacious and safe use, for example using ethylene oxide. When doing, it may be desirable to utilize a structure suitable for abatement of ethylene oxide that, for example, may be released from the medical device 18, the medical device package 12, etc. after sterilization. For example, FIG. 2 illustrate the medical device package 12 with a sterilizing gas abatement member 20. In this example, the sterilizing gas abatement member 20 may take the form of a pouch or sachet disposed within the medical device package 12. The sachet 20 may be disposed within the inner packaging member/pouch 14 as shown in FIG. 2. Alternatively, the sachet 20 may be disposed within the outer packaging member/pouch 16 as shown in FIG. 3 or both the inner and outer packaging members/pouches 14, 16 as shown in FIG. 4.

The sachet 20 may have a variety of different structural configurations. For example, the sachet 20 may comprise a pouch or container. The pouch may be formed from or include a polymeric material, fabric, and/or the like. In some instances, the sachet 20 may include a sterilizing gas abatement substance 28, represented by a speckled pattern in FIG. 2, disposed and/or incorporated therein. The sterilizing gas abatement substance 28 may be disposed within the sachet 20, for example as beads or the like. Alternatively, the sterilizing gas abatement substance 28 may be incorporated into the structure/fabric of the sachet 20. For example, the sachet 20 may include a woven fabric and the sterilizing gas abatement substance 28 may be woven into the fabric, for examples a one or more fibers woven into the fabric. A variety of alternatively arrangements and/or configuration are contemplated.

In some instances, the sachet 20 may include a material that is gas permeable while being substantially impermeable to moisture. For example, the sachet 20 may include a silicone material, expandable polytetrafluoroethylene, and/or other suitable materials. The use of materials that are gas permeable while being substantially impermeable to moisture may allow for a hydrated or liquid sterilizing gas abatement substance 28 to be disposed in the sachet 20. For example, some sterilizing gas abatement substances 28 may need to be hydrated in order to function optimally. For example, the use of chitosan as the sterilizing gas abatement substance 28 may include the use of hydrated chitosan. For a number of reasons including isolating a medical device from moisture, it may be desirable to dispose hydrated and/or liquid sterilizing gas abatement substances 28 in a sachet 20 that is gas permeable while being substantially impermeable to moisture.

The sterilizing gas abatement substance 28 may have a variety of forms. In general, the sterilizing gas abatement substance 28 may be configured to react with ethylene oxide gas and neutralize/abate the ethylene oxide gas. For example, the sterilizing gas abatement substance 28 may comprise a material that can bond (e.g., hydrogen bond) with ethylene oxide. In some instances, the sterilizing gas abatement substance 28 may comprise a polymer, for example a hydrophilic polymer. In some of these and in other instances, the sterilizing gas abatement substance 28 may be a nucleophile (e.g., a nucleophilic substance eager to donate electrons). In some of these and in other instances, the sterilizing gas abatement substance 28 may have a relatively basic or high pH (e.g., pH above 7). In some of these and in other instances, the sterilizing gas abatement substance 28 may include a sulfonated material/substance. In some of these and in other instances, the sterilizing gas abatement substance 28 may include an activated clay (e.g., such as an acid activated clay, bentonite, laponite, montmorillonite, combinations thereof, and/or the like). One example substance that may be used as the sterilizing gas abatement substance 28 may include chitosan. Another example substance that may be used as the sterilizing gas abatement substance 28 may include a lignosulfonic acid and/or lignocellulose. Another example substance that may be used as the sterilizing gas abatement substance 28 may include deacetylhyaluronic acid. In some instances, the sterilizing gas abatement substance 28 may include a material that can act as a catalyst for the neutralization of ethylene oxide. For example, the sterilizing gas abatement substance 28 may include sulfonated polystyrene. In some instances, polystyrene (e.g. sulfonated polystyrene) beads may be disposed within the sachet 20. In some of these and in other instances, the sterilizing gas abatement substance 28 may include a polyether ether ketone, a polyethylene, a polypropylene, combinations thereof, and/or the like.

In some instances, the sterilizing gas abatement member 20 can be selectively activated. For example, the sachet 20 may have the sterilizing gas abatement substance 28 therein. In an “unactivated” state, the sterilizing gas abatement substance 28 may be sealed within the sachet 20 and can be generally unavailable for reaction/abatement of ethylene oxide. A trigger such as a change in temperature and/or a change in pressure may open or activate the sachet 20, rendering the sterilizing gas abatement substance 28 available for reaction/abatement of ethylene oxide. For example, build-up/release of ethylene oxide gas within the medical device package 12 may increase the pressure within the medical device package 12. The increase in pressure can cause the sachet 20 to open, split, burst, and/or otherwise activate. In some instances, the sterilizing gas abatement member 20 can be in an activated state continuously. For example, the sterilizing gas abatement member 20 can or would react in the presence of ethylene oxide gas without an environmental (e.g., temperature) or physical change (e.g., pressure).

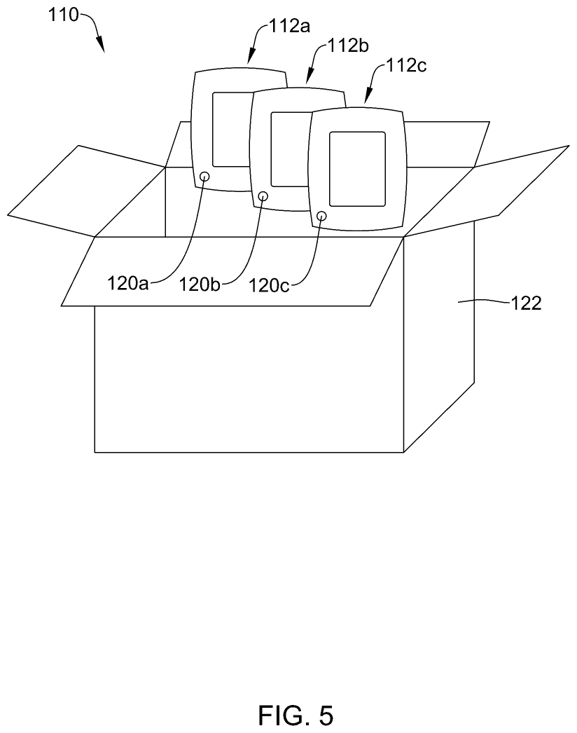

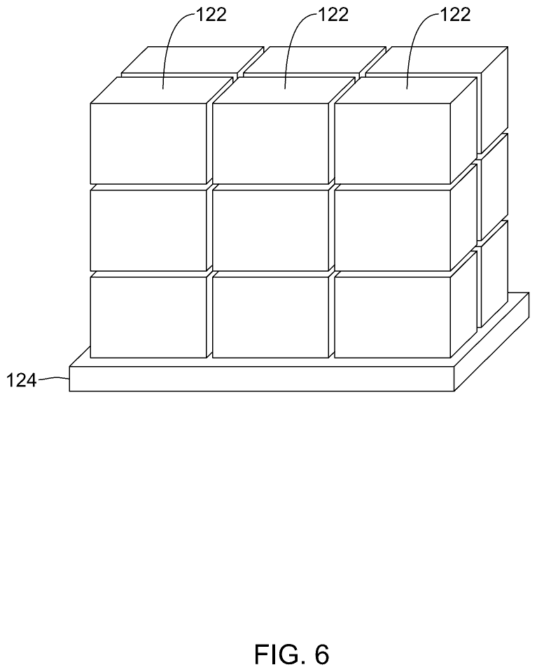

FIG. 5 illustrates a system 110 including a plurality of medical device packages 112a, 112b, 112c (e.g., where the medical device packages 112a, 112b, 112c have a sterilizing gas abatement member 120a, 120b, 120c disposed therein). The medical device packages 112a, 112b, 112c may be disposed within a container 122 such as box. In some instances, multiple containers 122 may be stacked together onto a pallet 124 as shown in FIG. 6. It can be appreciated that assemblies such as a plurality of medical device packages (e.g. medical device packages 112a, 112b, 112c), a plurality of medical device packages within a box/container 122, a plurality of boxes/containers 122 disposed on a pallet 124 could have the ability to release a relatively large amount of ethylene oxide gas. Disposing a sterilizing gas abatement member 20, for example within the medical device packages 112a, 112b, 112c) may significantly reduce the level of residual ethylene oxide gas present adjacent to such assemblies.

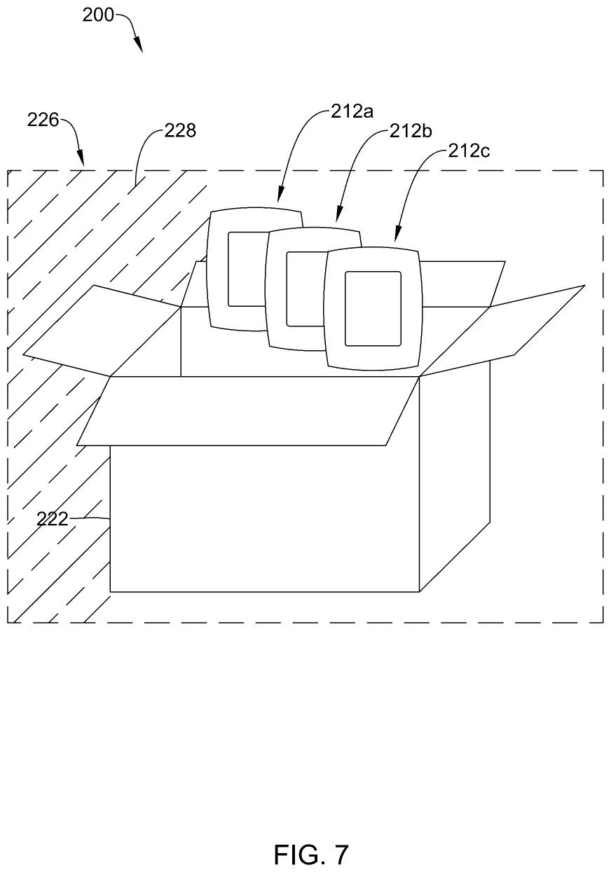

While the sterilizing gas abatement member 120a, 120b, 120c may be sufficient to abate residual ethylene oxide gas, other abatement structures are contemplated. For example, FIG. 7 illustrates a system 200 including a plurality of medical device packages 212a, 212b, 212c disposed within a container 222 such as box. In this example, a sterilizing gas abatement member 226 may be disposed along the container 222. The sterilizing gas abatement member 226 may take the form of a sterilizing gas blanket 226 having a sterilizing gas abatement substance 228 incorporated therein. Other form factors are contemplated for the sterilizing gas abatement member 226 includes wraps, boxes, containers, etc. with the sterilizing gas abatement substance 228 incorporated therein.

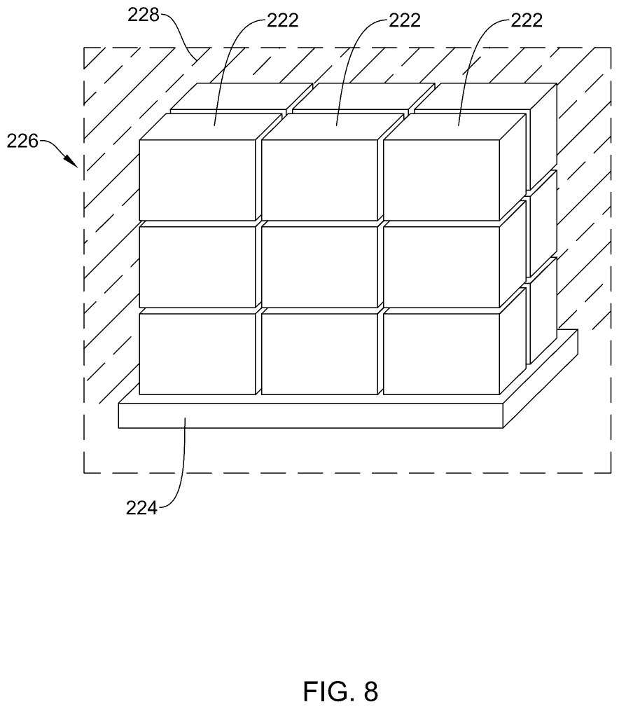

In some instances, the sterilizing gas abatement substance 228 may comprise a fibrous material and/or textile material that can be incorporated into the sterilizing gas blanket 226. For example, chitosan is a readily available, relatively inexpensive material (e.g., a byproduct of the seafood industry) that can be made into fibers and/or a textile material and that can be woven into the fabric of the sterilizing gas blanket 226. Other materials such as those disclosed herein may be incorporated into the sterilizing gas blanket 226. With the sterilizing gas abatement substance 228 woven/incorporated into the fabric of the sterilizing gas blanket 226, the sterilizing gas blanket 226 can be disposed on, about, form-fitted, etc. on various assemblies of medical device packages. For example, the sterilizing gas blanket 226 can be disposed about a singular medical device package 212a, 212b, 212c. In some of these and in other instances, the sterilizing gas blanket 226 can be disposed about a box or container 222 having a plurality of medical device packages 212a, 212b, 212c disposed therein (e.g., as shown in FIG. 7). In some instances, multiple containers 222 may be stacked together onto a pallet 224 as shown in FIG. 8. In such instances, the sterilizing gas blanket 226 may be disposed along, onto, or over the pallet 224 and containers 222 to abate ethylene oxide gas.

In some of these and in other instances, the sterilizing gas blanket 226 may include metallic and/or polymeric beads (e.g., relatively small beads) may be incorporated into the sterilizing gas blanket 226. In some of these and in other instances, the sterilizing gas blanket 226 may include a hybrid material of frit including particulates or fused particles that gasses can pass through for adsorption/reaction. Other forms are contemplated.

The sterilizing gas blanket 226 can suitably scavenge ethylene oxide gas that may diffuse from the medical device packages (e.g., medical device packages 212a, 212b, 212c). The sterilizing gas blanket 226 may have a suitable surface area to cover the medical device packages (e.g., medical device packages 212a, 212b, 212c), boxes or containers 222 (e.g., having a plurality of medical device packages 212a, 212b, 212c disposed therein, multiple boxes or containers 222 stacked together onto a pallet 224, etc. In addition, the woven nature of the sterilizing gas blanket 226 (e.g., when such a form factor is utilized) may expand/increase the surface area onto which ethylene oxide can interact with the fabric and, in particular, the sterilizing gas abatement substance 228.

It can be appreciated that sterilizing gas blankets 226 and sachets 20 can be used together or separately. For example, it is contemplated that one or more medical device packages may include a sachet 20. Assemblies of such medical devices can be draped with a sterilizing gas blanket 226.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for abatement of sterilizing gas, the system comprising:
a medical device package having a medical device disposed therein; and
a sterilizing gas abatement member disposed adjacent to the medical device, the sterilizing gas abatement member being configured to abate a sterilizing gas present adjacent to the medical device.

2. The system of claim 1, wherein the medical device comprises a sterilized medical device.

3. The system of claim 1, wherein the sterilizing gas comprises ethylene oxide.

4. The system of claim 1, wherein the sterilizing gas abatement member includes a sachet.

5. The system of claim 4, wherein the sachet is disposed within the medical device package.

6. The system of claim 4, wherein the medical device package includes an inner pouch and an outer pouch, and wherein the sachet is disposed within the inner pouch.

7. The system of claim 4, wherein the medical device package includes an inner pouch and an outer pouch, and wherein the sachet is disposed within the outer pouch.

8. The system of claim 1, wherein the sterilizing gas abatement member includes a blanket.

9. The system of claim 8, wherein the blanket is disposed about the medical device package.

10. The system of claim 8, further comprising one or more additional medical device packages, and wherein the blanket is disposed about the medical device package and the one or more additional medical device packages.

11. The system of claim 1, further comprising a sterilizing gas abatement substance incorporated within the sterilizing gas abatement member.

12. The system of claim 11, wherein the sterilizing gas abatement substance includes a hydrophilic material.

13. The system of claim 11, wherein the sterilizing gas abatement substance includes a hydrophilic polymer.

14. The system of claim 11, wherein the sterilizing gas abatement substance includes a nucleophilic material.

15. The system of claim 11, wherein the sterilizing gas abatement substance includes chitosan.

16. The system of claim 11, wherein the sterilizing gas abatement substance includes a lignosulfonic acid.

17. A system for abatement of residual ethylene oxide, the system comprising:
a medical device package having a sterilized medical device disposed therein;
wherein the medical device package includes an inner package, an outer package, or both;
an ethylene oxide abatement sachet disposed adjacent to the sterilized medical device, the sachet having an ethylene oxide abatement substance disposed therein; and
wherein the sachet is configured to abate residual ethylene oxide present adjacent to the sterilized medical device.

18. The system of claim 17, wherein the ethylene oxide abatement substance includes a hydrophilic material, a hydrophilic polymer, a nucleophilic material, chitosan, a lignosulfonic acid, or combinations thereof.

19. A system for abatement of residual ethylene oxide, the system comprising:
a medical device package having a sterilized medical device disposed therein;
wherein the medical device package includes an inner package, an outer package, or both;
an ethylene oxide abatement blanket disposed adjacent to the sterilized medical device, the blanket having an ethylene oxide abatement substance incorporated therein; and
wherein the blanket is configured to abate residual ethylene oxide present adjacent to the sterilized medical device.

20. The system of claim 19, wherein the ethylene oxide abatement substance includes a hydrophilic material, a hydrophilic polymer, a nucleophilic material, chitosan, a lignosulfonic acid, or combinations thereof.
